(19) United States
(12) Patent Application Publication
(10) Pub. No.: US 2022/0390416 A1
(43) Pub. Date: Dec. 8, 2022

(54) EDDY CURRENT INSPECTION OF METAL CONTAINERS

(71) Applicant: BALL CORPORATION, WESTMINSTER, CO (US)

(72) Inventor: John D. EFNER, BROOMFIELD, CO (US)

(21) Appl. No.: 17/774,579

(22) PCT Filed: Nov. 6, 2020
(86) PCT No.: PCT/US2020/059362

§ 371 Date: May 5, 2022

Related U.S. Application Data
(60) Provisional application No. 62/932,645, filed on Nov. 8, 2019.

(51) Int. Cl. G01N 27/9013; G01N 33/2045

(52) U.S. Cl. CPC G01N 27/9026; G01N 33/2045

(57) ABSTRACT

A method of inspecting metallic container components (10) provides indexing a container component (10) produced from a metallic material into axial alignment with a probe (110). The probe (110) has a coil (176) produced from an electrical conductor. An alternating current (183) is applied to the coil (176) wherein a first magnetic field (184) is generated. An eddy current (188) develops in the container component (10) in response to the first magnetic field (184). A second magnetic field (192) is generated in response to the eddy current (188). Changes in an impedance amplitude and phase angle in the coil (176) are measured. A determination of the fitness for use of the container component (10) is made based on the measured changes in the impedance amplitude and phase angle in the coil (176).

### CROSS-REFERENCE TO RELATED APPLICATIONS

This application claims the benefit of U.S. Provisional Patent Application No. 62/932,645, filed on Nov. 8, 2019, and hereby incorporated by reference as if fully set forth herein.

### FEDERALLY SPONSORED RESEARCH OR DEVELOPMENT

N/A

### TECHNICAL FIELD

The invention relates to metal containers; more particularly, the invention relates to inspection of upper portions of metal containers.

### BACKGROUND

Containers produced from metallic materials are becoming increasingly popular for many reasons, including their versatility and carbon footprint compared to similar body s produced from other materials. These containers are typically produced from an aluminum alloy because aluminum alloys tend to form well and are capable of maintaining adequate strength even when drawn and ironed down to a very thin thickness. Metallic containers are used throughout the consumer product industry, including but not limited to the beverage industry and the aerosol industry. These containers generally include a lower portion or body having an enclosed bottom and a cylindrical sidewall extending upwardly from the enclosed bottom portion.

The bottom has a dome-shaped center panel surrounded by a generally circumferential annular support. An outer wall extends radially outwardly and upwardly relative to the annular support and joins the bottom with the lowermost portion of the cylindrical sidewall.

The cylindrical sidewall is centered about the longitudinal axis. The sidewall is generally smooth and flat; however, any one of a number of forming techniques can be employed to impart a shape and/or texture to the sidewall. For instance, the interior of the sidewall could be forced outwardly by a fluid pressure or forming segments, laser treatment could be employed to etch or otherwise mark the sidewall, and/or flutes or other designs may be imparted onto the sidewall through mechanical deformation of the sidewall.

An upper portion of the container body includes a circumferential shoulder portion. The shoulder has a convexly curved appearance when viewed from a vantage point external to the container body. The shoulder has a lowermost point integral with an uppermost portion of the cylindrical sidewall. The transition point between the sidewall and shoulder is at a point where the container body begins to curve radially inwardly. Stated another way, the diameter of the container body begins to decrease at the point where the shoulder begins and the sidewall ends.

The upper portion further includes an inwardly tapered circumferential neck. The neck has a lowermost portion integral with an uppermost portion of the shoulder. Thus, the neck functions to further decrease the diameter of the container body along the vertical length of the neck.

The upper portion terminates at a curl or a flange which is adapted for receiving a cap or closure member, including, for example, beverage can ends and aerosol valve assemblies.

During the forming of the container body neck. small wrinkles may form on or near the open end. These wrinkles may be pressed out during subsequent necking operations by forcing the edge of the can between the cylindrical upper portion of the necking die and the floating pilot member. The pressed out wrinkles create localized regions exhibiting increased work hardening that are generally more brittle than adjacent areas and may fail (i.e. fracture or crack) when the open end is flanged.

Wrinkles become even more prevalent as the container sidewall is down-gauged. To avoid wrinkling, four to six additional necking operations may be required. Additional necking operations, however, require additional manufacturing space, pressurized air, electricity, and manufacturing time. Thus, adding additional necking operations is cost prohibitive.

Inspection of the open ends of containers is generally performed by visual means, either by human eye or by an imager, such as a still or video camera.

In a further manufacturing process, the internal surfaces of container bodies are sprayed a fluid, which, when cured, forms a continuous protective coating on the internal surfaces. Internal coating is applied before necking, flanging and curling and can serve as a lubricant between the inside surface of the can and the tooling.

The present invention is provided to solve the problems discussed above and other problems, and to provide advantages and aspects not provided by prior inspection container body inspection of this type. A full discussion of the features and advantages of the present invention is deferred to the following detailed description, which proceeds with reference to the accompanying drawings.

### SUMMARY

A first aspect of the present invention is directed to a method of inspecting metallic container components comprising the steps of:
(i) transferring a container component produced from a metallic material into axial alignment with a probe, the probe comprising a coil produced from an electrical conductor;
(ii) applying an alternating current to the coil wherein a first magnetic field is generated;
(iii) developing an eddy current in the container component in response to the first magnetic field;
(iv) generating a second magnetic field from the eddy current;
(v) measuring changes in an impedance amplitude and phase angle in the coil; and
(vi) making a determination of the fitness for use of the container component based on the measured changes in the impedance amplitude and phase angle in the coil.

The first aspect of the invention may include one or more of the following features, alone or in any reasonable combination. The method may further comprise the step of rotating the container component about a central axis of the container component during the step of developing the eddy current in the container component. The method may further comprise the step of altering an axial distance between the probe and the container component along a linear direction parallel to the central axis of the container component. The method may further comprise the step of linearly moving the probe along the linear direction parallel to the central axis of the container component. The method may further comprise the step of providing a controller including a first software routine stored on a non-transitory computer readable medium wherein the first software routine outputs a proper level and timing of the alternating current determines the control. The method may further comprise the step of analyzing the measured changes in the impedance amplitude and phase angle using a second software routine stored in the non-transitory computer readable medium of the controller. The method may further comprise the step of providing a rotational indexer having a plurality of vacuum chucks spaced about a circumference of the indexer wherein the container component is retained to the indexer by a corresponding vacuum chuck. A rotatable turntable may be associated with each vacuum chuck such that the container component is retained to one turntable by a vacuum force delivered by the vacuum chuck. Each rotatable turntable may be engaged by a rotational belt which imparts rotation to the turntable. The container component may be a container body. The container body may comprise an open end having a circumferential curl about an opening. The eddy current may be located within the circumferential curl.

A second aspect of the present invention is directed to a metallic container component testing apparatus comprising:
(i) a probe comprising a coil of an electrically conductive material;
(ii) an alternating electric current transmitted to the coil;
(iii) a rotational turntable configured to retain a container component thereto and rotate about a center axis of the container component; and
(iv) an impedance analyzer.

The second aspect of the invention may include one or more of the following features, alone or in any reasonable combination. The metallic container component testing apparatus may further comprise a controller wherein the controller has at least one software subroutine stored on a non-transitory computer readable medium wherein output from the software routine controls a level and timing of the alternating current. The metallic container component testing apparatus may further comprise a rotational indexer wherein a plurality of rotational turntables are located about a circumference of the indexer and attached thereto such that the indexer imparts an orbit to each turntable about a central axis of the indexer. The metallic container component testing apparatus may further comprise a plurality of vacuum chucks, each vacuum chuck associated with one turntable in the plurality of turntables wherein a source of a vacuum pressure delivers a vacuum force to each vacuum chuck to retain the container component to each turntable. The metallic container component testing apparatus may further comprise a rotational belt wound about a plurality of pulleys and engaging at least one turntable in the plurality of turntables to impart rotation to the at least one turntable. The metallic container component testing apparatus may further comprise a first servo motor coupled to the indexer and imparting rotation thereto. The metallic container component testing apparatus may further comprise a second servo motor coupled to the probe wherein the second servo motor imparts a movement to the probe to control a distance between the probe and the container component along a path parallel to a center axis of the container component. The second servo motor may be a linear servo motor. An output signal from the controller to the second servo motor may control the distance between the probe and the container component. An output signal from the controller to the first servo motor may control indexing of the container components.

Other features and advantages of the invention will be apparent from the following specification taken in conjunction with the following drawings.

### BRIEF DESCRIPTION OF THE DRAWINGS

To understand the present invention, it will now be described by way of example, with reference to the accompanying drawings in which:

FIG. 1 is a front view of an inspection apparatus of the present invention;

FIG. 2 is a front view of an inspection apparatus showing a spinner belt with a probe and an exit chute removed;

FIG. 3 is a top view of an inspection station of the present invention;

FIG. 4 is a top view of an inspection station showing a lancing servo to provide relative movement between a probe and an open end of a container body;

FIG. 5 is a magnified view of an inspection of a metal container via a detecting method of the present invention, a bolded arrow indicates container rotation during inspection;

FIG. 6 is a graphical representation of a positive inspection test result;

FIG. 7 is a graphical representation of a negative inspection test result; and

FIG. 8 is a graphical representation of a negative inspection test result wherein the detected defect is physically smaller than the defect detected in the test represented in FIG. 7.

### DETAILED DESCRIPTION

While this invention is susceptible of embodiments in many different forms, there is shown in the drawings and will herein be described in detail preferred embodiments of the invention with the understanding that the present disclosure is to be considered as an exemplification of the principles of the invention and is not intended to limit the broad aspect of the invention to the embodiments illustrated.

The present invention is directed to a method and apparatus of detecting metal flaws in formed metal containers using a controlled magnetic field to form eddy currents within a targeted area. An alternating current flowing through a coil at a chosen frequency generates a magnetic field around the coil. When the coil is placed close to an electrically conductive material, an eddy current is induced in the material. If a flaw in the conductive material disturbs the eddy current circulation, a magnetic coupling with a probe is changed and a defect signal can be read by measuring impedance variation in the coil. This method and apparatus can be used to detect fractures, cracks, splits, tears, voids, etc. in curls and flanges located at an open end of metallic container bodies.

The present invention augments or replaces present inspection systems that employ imagers, such as cameras, to detect manufacturing defects. The current systems use software to interpret images captured by the imager for finding flaws. By using an electrical current rather than images, actual manufacturing defects can be identified though a disruption in a path of electron flow.

The present disclosure may be incorporated into a forming system, station, or apparatus to detect cracks on a curl of metal defining a pour opening of a metallic bottle. As bottles are formed, occasionally the curl at the open end of the bottle with crack, split fracture, etc. Present detection methods include using a camera in conjunction with software to determine if a crack is present. The present disclosure includes using eddy currents which do not rely on software to interpret a picture/photograph. The eddy current is injected into the metal, reacts to deformities in the curl, and returns to signal to a sensor if/when there is a physical defect present.

Eddy current inspection is based on electromagnetic induction. In an eddy current probe, an alternating current flows through a wire coil and generates an oscillating magnetic field. If the probe and its magnetic field are brought close to a conductive material like aluminum, a circular flow of electrons known as an eddy current will begin to move through the metal, similar to a swirling water in a stream. That eddy current flowing through the metal will in turn generate its own magnetic field, which will interact with the coil and its field through mutual inductance. Changes in metal thickness or defects, like surface cracking will interrupt or alter the amplitude and pattern of the eddy current and the resulting magnetic field. This in turn affects the movement of electrons in the coil by varying the electrical impedance of the coil.

A processor or eddy current instrument plots changes in the impedance amplitude and phase angle, which can be used to reject a defected container component, such as a container body.

The requirement is to have the product be suspended with the exposed portion of the product spinning. As the machine indexes and spins the container, the sensor will emit the necessary currents to test for defects.

The container will load into the apparatus and be held horizontally by a vacuum chuck. The turntable is one of 6 on a plate which indexes in a circle. While the plate indexes the turntable is spinning 2 to 3 revolution per index step. When the indexer moves/rotates, the turntable holding the container with vacuum in front of the sensor, the magnetic field will induce the eddy current into the container, any flaws in the metal will be sensed and rejected. It is contemplated that the container can be held vertically as well by altering the orientation of the turntables and associated apparatus elements.

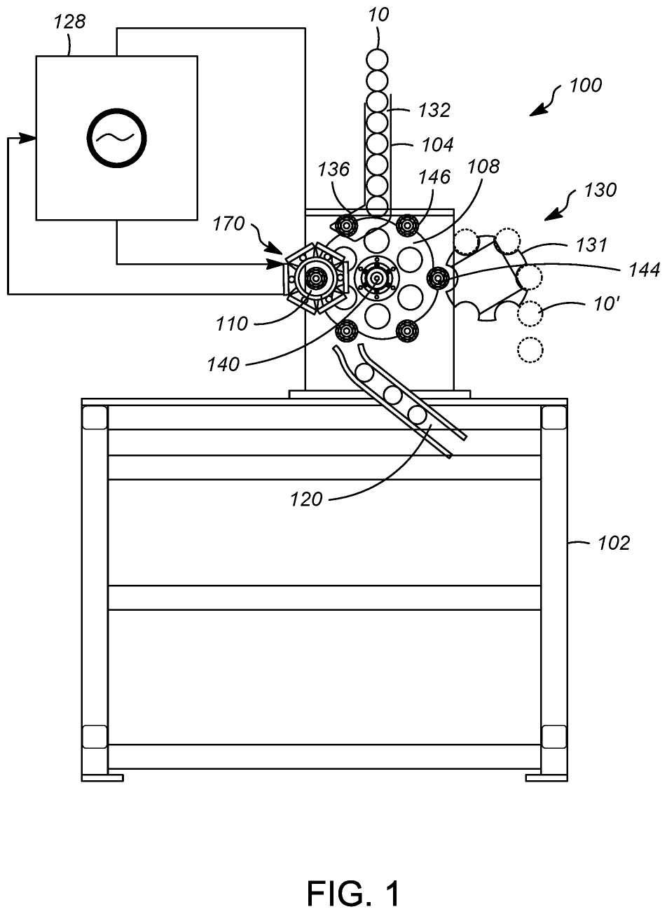

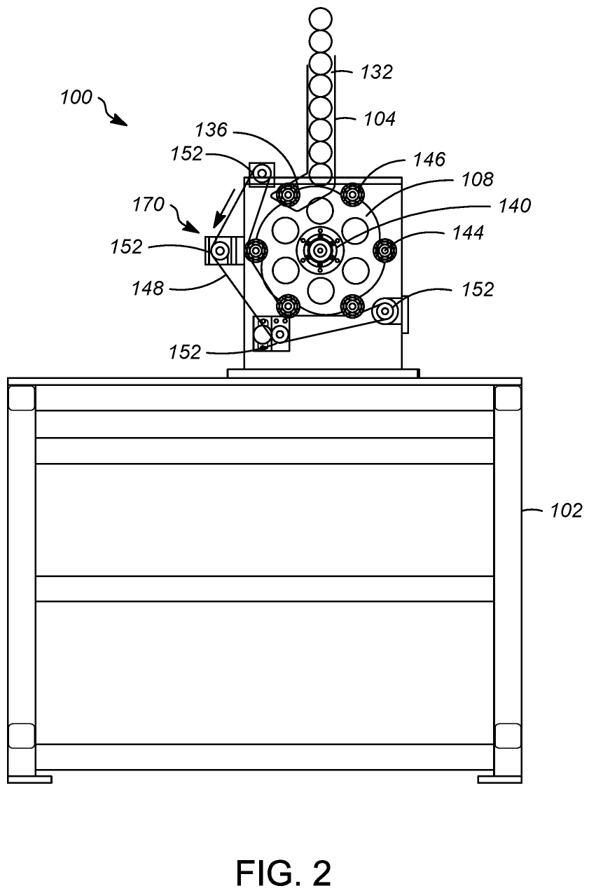

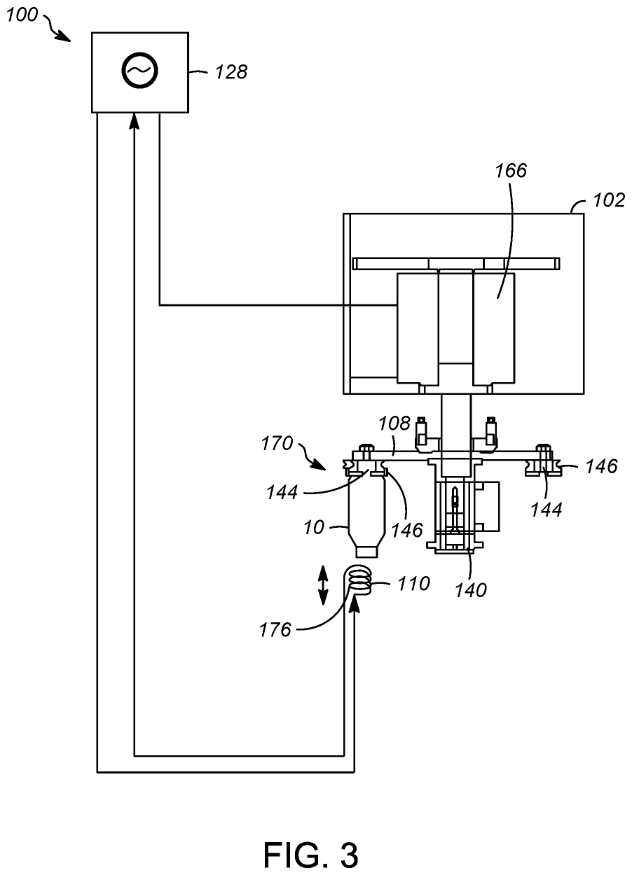

Now, referring to FIGS. 1-4, a container body inspection apparatus 100 is supported by a frame 102 and generally includes a container body feeder 104, an indexing means, such as a rotational indexer 108, an eddy current probe 110, and a container body delivery chute 120. A programmable controller 128 is also connected to the apparatus, the purpose of which will become clear upon further description. This type of container body inspection apparatus is capable of inspecting the manufacturing quality of the formed metal of the container instantaneously or at least substantially instantaneously. The limitation on inspection rate, therefore, is associated with the container handling equipment.

The frame 102 is suitable for supporting, and attachment to, the various components that make up the apparatus 100. Accordingly, the frame 102 has a plurality of rigid members, generally produced from a metal such as a steel, and configured to reduce movement of the machine caused by processing of container bodies.

The indexing means sequentially transfers a plurality of container bodies 10 along a predetermined fixed path through the inspection process and into an out of axial alignment with the probe 110. In the apparatus illustrated in FIGS. 1-3, an indexer 108 includes a rotary plate having a plurality of pockets 136 which rotates about a hub 140 at a central axis of the indexer 108. Any number of pockets can be provided as feasibly possible. In the apparatus 100 illustrated in FIGS. 1-4, there are six (6) pockets 136 forming a 60 degree index. The inventors contemplate that the apparatus disclosed herein may be provided with a 30 degree index, a 60 degree index, or any other degree index without departing from the spirit of the invention. In other words, one indexing means as contemplated herein comprises a plurality of equally spaced index positions about a circumference of a rotational indexer.

Increasing the number of pockets can lead to increased production. For example, one advantage of the 12-pocket apparatus is that it may be used to process two container bodies 10 simultaneously by indexing 60 degrees, thus doubling the production rate.

Each pocket 136 has a vacuum chuck 144. The vacuum chucks 144 utilize a vacuum pressure or force to maintain the container bodies 10 in position as the indexer 108 indexes the container bodies 10 through the inspection process. Thus, the vacuum chucks 144 are each in fluid communication with a source of fluid pressure. The vacuum pressure is used to attach each container body 10 to the indexer 108. The vacuum chucks 144 have been used in the art of container manufacturing for many years.

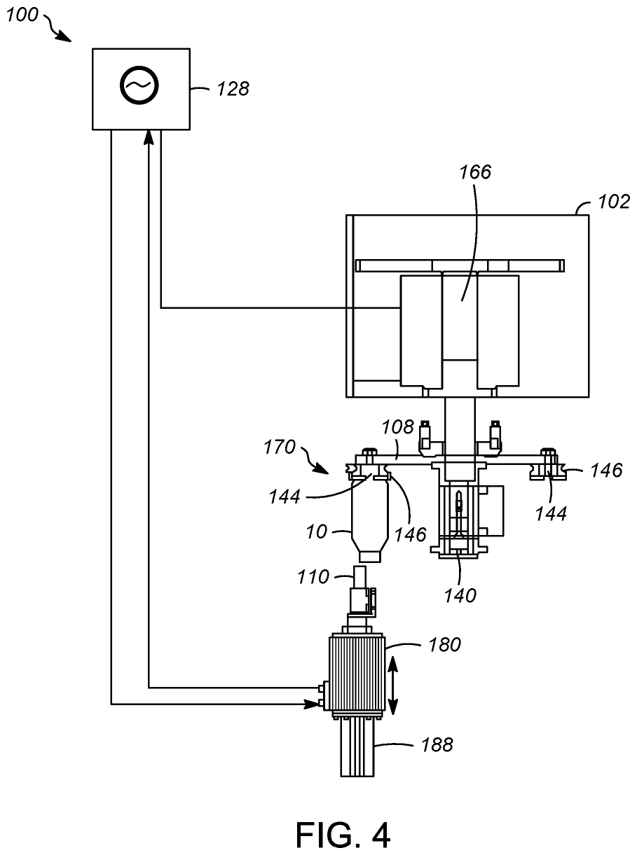

A rotatable turntable 146 is associated with each vacuum chuck 144. The turntables 146 are substantially free-wheeling. According to FIG. 4, this enables a spinner belt 148 wound around a plurality of idler pulleys 152 to impart rotational movement to the turntables 146 which in turn imparts a rotation to the container bodies 10 about a center axis of the container bodies 10 attached to the vacuum chucks 144. One of the idler pulleys 152 is operably joined to a spinner motor which drives the spinner belt 148. One or more spinner gears may be provided to control the revolutions per minute of the container bodies 10.

The programmable controller 128 is typically external to the frame 102. It includes a non-transitory computer readable medium whereon one or more software subroutines can be stored for execution by a processor also included with the controller. The controller 128 is in communication with a source of vacuum pressure and the spinner motor. It can be used to signal a response in source of vacuum pressure to activate and deactivate the vacuum force in one or more vacuum chucks 144, individually and/or in combination. Likewise, activation and rate of the rotation of the turntables 146 can also be controlled by a software subroutine on the controller 128.

Container bodies 10 exit the apparatus 100 via the delivery chute 120 for further processing, packaging and delivery, filling, etc.

Rejected container bodies 10′, shown is dashed lines in FIG. 1, can be identified and culled from the production queue via a rejection module 130. In illustrated embodiment, when a container body 10′ does not meet manufacturing specifications, it is identified during the inspection and bypasses the delivery chute 120 as a corresponding vacuum chuck 144 remains activated until the container body 10′ arrives at a rejection position along the indexer 108. The vacuum chuck 144 is then deactivated, and the container body 10′ is removed by a rotary handler 131.

The apparatus 100 of FIGS. 1-4 further includes a first servo drive motor 166. The first servo drive motor 166 is operably connected to the indexer 108 via a shaft 168 projecting outwardly from the indexer hub 140 into the first servo drive motor 166.

Thus, according to the described structure, the indexer 108 is driven by the first servo drive motor 166. This causes the indexer 108 to rotate about the hub 140. Correspondingly, the pockets 136, the vacuum chucks 144, and the turntables 146 rotate about the hub 140 with the rotation of the indexer. The turntables 146 further rotate about their axes of rotation which are generally coincident with the center axis of the container bodies 10. In other words, the turntables 146 orbit the hub 140 while rotating about their own axes of the rotation.

The controller 128 is in communication with the first servo motor 166. It can be used to signal a response in the first servo motor 166 to any predetermined dwell time independent of the speed of the indexer 108, which is also controlled by a software subroutine on the controller 128. Thus, this apparatus 100 can be programmed based on time without mechanical intervention.

The apparatus 100 further includes the probe 110. The probe 110 comprises a coil 176 produced from an electrical conductor, typically a bare wire. In the apparatus of FIG. 1, the probe 110 is joined to the frame 102 and mounted at the 60 degree index position or 9 o'clock position in a clock-like orientation. This defined the inspection position 170 of the apparatus which the indexer 108 transports container bodies 10 to and from, or into and out of, while the container bodies 10 are attached to the turntables 146 by the vacuum chucks 144. The probe 110 is generally about the width of an ordinary pencil, about 6 mm to 7 mm wide. The probe 110 shown in the drawings is somewhat not to scale in order to show the structure involved.

As shown in FIG. 4, the probe 110 is capable of relative movement between the indexer 108 wherein a distance between the indexer 108 and the probe 110 may be controlled, i.e. reduced or enlarged. Thus, the probe 110 is capable of movement relative to a container body 10 adhered to the indexer 108 at the inspection position 170 of the apparatus 100. This movement is preferably a linear movement to traverse the probe 110 from a first position to a second position adjacent the open end 14 of the container body 10. The linear movement is illustrated by arrows and is preferably substantially parallel to the container axis. Regardless, the movement should be perpendicular to an imaginary plane defined by the opening of the container body 10 and/or along a straight line parallel to the center axis of the container body 10, coincident with the axis of rotation of the turntables 144 when aligned therewith. Typically, this imaginary plane is a vertical plane. Movement of the probe 110 is accomplished by operably connecting or coupling the probe 110 to a second servo motor 180, preferably a lancing servo motor. The probe 110 is attached to a guide 182 controlled, preferably directly controlled, by its corresponding second servo motor 180.

The programmable controller 128 also controls the probe 110 movement via signaling to the second servo motor 180.

Further to the feeding of container bodies, container bodies 10 enter the apparatus 100 via the feeder 104. Gravity acts to transfer the container bodies 10 one-by-one through an entry chute 132 which delivers the container bodies 10 to the indexer 108.

In use, container bodies 10 index counterclockwise on the indexer 108 in 60 degree increments. A dwell time is established at each index position. The dwell time can be varied as desired to any predetermined duration via controller 128. This dwell time is typically restricted by the rate at which the inspection of a container body 10 can be performed. Some additional time may be added to account for any necessary movement by the probe 110. However, this is dependent upon the nature of the container body 10 to be inspected. The expected dwell time being about 200 ms, preferably within the range of 50 ms to 500 ms, more preferably 100 ms to 400, and most preferably about 150 ms to 250 ms, or any value, range or combination of ranges therein.

It should be understood that the apparatus 100 is programmable, and any number of dwell time preferences can be achieved on the same apparatus 100 without the need for mechanical changes to the apparatus 100. Thus, the relative movement between the probe and the indexing means is both selective and automatic as controlled by a signal delivered from the external programmable controller 128 the probe 110. Furthermore, the external programmable controller 128 can be used to selectively alter the current.

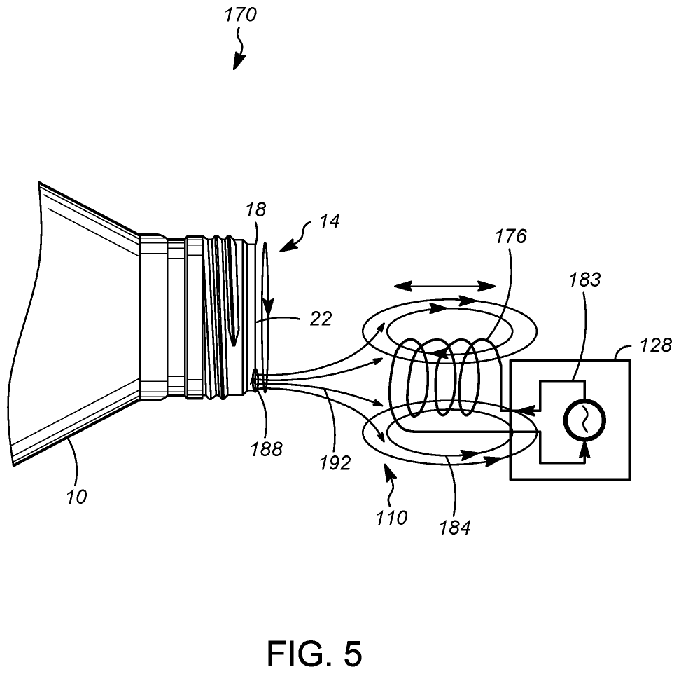

As best illustrated in FIG. 5, in operation, at the inspection position 170, the probe 110 is brought into close proximity with the open end 14 of the container body 10 and, preferably, with a circumferential curl 18 defining an opening 22 in the open end 14. An alternating current 183 (shown by arrows) is applied to the coil 176. An oscillating first magnetic field 184 is generated adjacent the coil 176. This causes an eddy current 188 to develop within the metallic material in the open end of the container body 10, similar to a swirling water in a stream. A secondary or eddy current magnetic field 192 is generated. The direction of the eddy current 188 is such that its magnetic field 192 interacts with the coil 176 and its magnetic field 184 through mutual inductance, opposing the magnetic field 184 of the coil 176. Manufacturing defects and variances in metal thickness of the container body 10 cause variations in the electrical conductivity and magnetic permeability of the container body 10 which changes the eddy current 188 and the impedance seen by the coil 176. These anomalies in the container body material, like surface cracks, holes, splits, etc., interrupt or alter the amplitude and pattern of the eddy current 188 and the resulting magnetic field 184. This in turn affects the movement of electrons in the coil 176 by varying the electrical impedance of the coil 176. An oscilloscope or other electrical property measuring or analyzing device, e.g. an impedance analyzer, measures changed in the coil 176 and a processor, e.g. within the controller 128, monitors and/or plots changes in the impedance amplitude and phase angle. This information is used by a subroutine stored in the non-transitory computer readable medium to output a signal corresponding to a pass/fail analysis and comparison of the measured electrical property(ies) against a predetermined manufacturing quality standard. A further software subroutine, similarly stored, can control activation and deactivation of the vacuum chucks 144 and operation of the rejection module 130 to cull rejected container bodies 10′ from the manufacturing queue.

It should be understood that the functionality of the apparatus 100 is electrically controlled by the controller 128. Physical movements in terms of timing, degree of displacement, duration, etc. are coordinated by one or more software subroutines stored on one more non-transitory computer readable medium. Further, activation, timing, level, and duration of the alternating current supplied to the probe 108 are also coordinated by one or more software subroutines stored on one more non-transitory computer readable mediums included with the controller 128. It follows that the controller 128 may incorporate a computer or other suitable electronic device capable of storing and executing software routines in electrical communication with various sources devices which serve, control, and provide the functionality of the apparatus.

Additionally, in a broad sense, the apparatus disclosed herein can be retrofit on existing bottle forming and handling equipment. For example, currently, the detection practice includes mechanical elements which provide a rotational movement to a bottle 10 while a camera inspects takes and collects images of the bottle 10. The eddy current probe 110 described herein can be mounted within this equipment, and no additional handling equipment would be necessary.

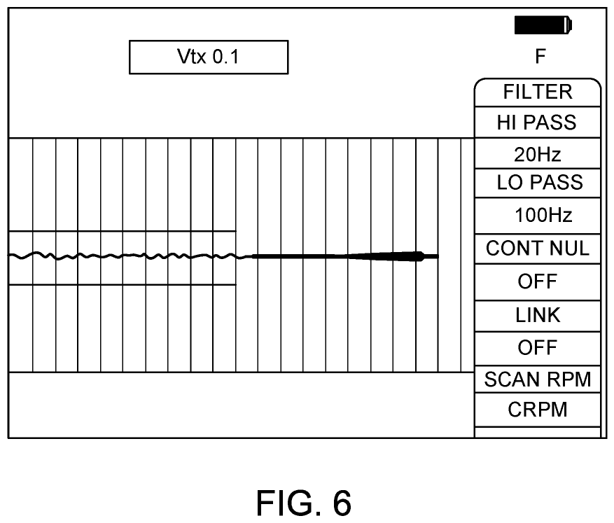

Referring to FIGS. 6-8, testing performed according to the present disclosure showed the technology can detect small cracks in the curl 18 while the metallic bottle 10 was rotating, similar to how the manner in which such metallic bottles 10 are transported in a production line. The testing confirmed the probe 110 is able to discern accurately the difference between a good curl 18 and a defective or cracked curl 18.

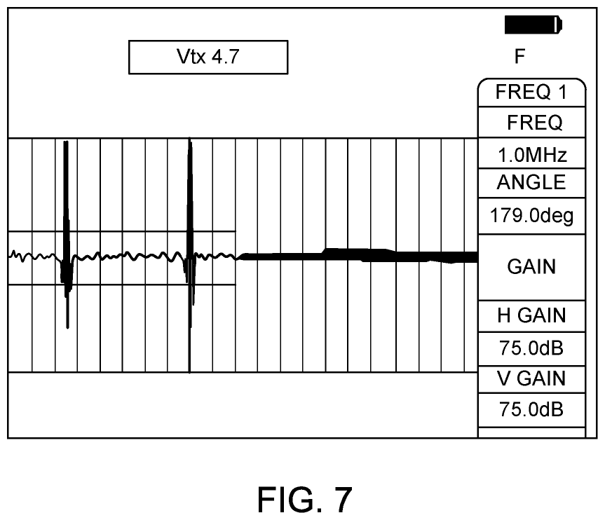

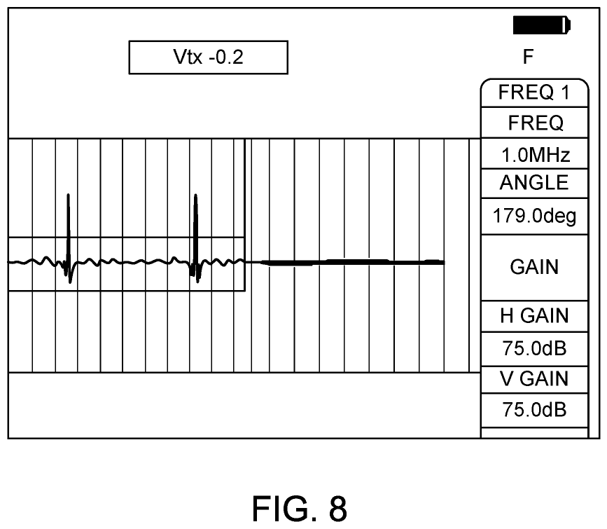

During the tests, multiple bottles 10 were provided with varied defect sizes. The probe 110 displayed varying signals representing the defect size. For example, FIG. 6 shows the result for a non-defective bottle 10; FIG. 7 shows the result for a bottle with a larger defect; and FIG. 8 shows the result for a bottle with a smaller defect.

While the specific embodiments have been illustrated and described, numerous modifications come to mind without significantly departing from the spirit of the invention, and the scope of protection is only limited by the scope of the accompanying Claims.

1-24. (canceled)

25. A method of inspecting metallic container components comprising the steps of:
indexing a container component produced from a metallic material into axial alignment with a probe, the probe comprising a coil produced from an electrical conductor;
applying an alternating current to the coil wherein a first magnetic field is generated;
developing an eddy current in the container component in response to the first magnetic field;
generating a second magnetic field from the eddy current;
measuring changes in an impedance amplitude and phase angle in the coil; and
making a determination of the fitness for use of the container component based on the measured changes in the impedance amplitude and phase angle in the coil.

26. The method of claim 25 further comprising the step of rotating the container component about a central axis of the container component during the step of developing the eddy current in the container component.

27. The method of claim 26 further comprising the step of altering an axial distance between the probe and the container component along a linear direction parallel to the central axis of the container component.

28. The method of claim 27 further comprising the step of linearly moving the probe along the linear direction parallel to the central axis of the container component.

29. The method of claim 25 further comprising the step of providing a controller including a first software routine stored on a non-transitory computer readable medium wherein the first software routine outputs a proper level and timing of the alternating current.

30. The method of claim 25 further comprising the step of analyzing the measured changes in the impedance amplitude and phase angle using a second software routine stored in the non-transitory computer readable medium of the controller.

31. The method of claim 30 further comprising the step of providing a rotational indexer having a plurality of vacuum chucks spaced about a circumference of the indexer wherein the container component is retained to the indexer by a corresponding vacuum chuck.

32. The method of claim 31 wherein a rotatable turntable is associated with each vacuum chuck such that the container component is retained to one turntable by a vacuum force delivered by the vacuum chuck.

33. The method of claim 32 wherein each rotatable turntable is engaged by a rotational belt which imparts rotation to the turntable.

34. The method of claim 25 wherein the container component is a container body.

35. The method of claim 34 wherein the container body comprises an open end having a circumferential curl about an opening.

36. The method of claim 35 wherein the eddy current is located within the circumferential curl.

37. A metallic container component testing apparatus performing the method of claim 1 comprising:
a probe comprising a coil of an electrically conductive material;
an alternating electric current transmitted to the coil;
a rotational turntable configured to retain a container component thereto and rotate about a center axis of the container component; and
an impedance analyzer.

38. The metallic container component testing apparatus of claim 37 further comprising a controller wherein the controller has at least one software subroutine stored on a non-transitory computer readable medium wherein output from the software routine controls a level and timing of the alternating current.

39. The metallic container component testing apparatus of claim 38 further comprising a rotational indexer wherein a plurality of rotational turntables are located about a circumference of the indexer and attached thereto such that the indexer imparts an orbit to each turntable about a central axis of the indexer.

40. The metallic container component testing apparatus of claim 39 further comprising a plurality of vacuum chucks, each vacuum chuck associated with one turntable in the plurality of turntables wherein a source of a vacuum pressure delivers a vacuum force to each vacuum chuck to retain the container component to each turntable.

41. The metallic container component testing apparatus of claim 40 further comprising a rotational belt wound about a plurality of pulleys and engaging at least one turntable in the plurality of turntables to impart rotation to the at least one turntable.

42. The metallic container component testing apparatus of claim 41 further comprising a first servo motor coupled to the indexer and imparting rotation thereto.

43. The metallic container component testing apparatus of claim 42 further comprising a second servo motor coupled to the probe wherein the second servo motor imparts a movement to the probe to control a distance between the probe and the container component along a path parallel to a center axis of the container component.

44. The metallic container component testing apparatus of claim 43 wherein the second servo motor is a linear servo motor.

45. The metallic container component testing apparatus of claim 44 wherein an output signal from the controller to the second servo motor controls the distance between the probe and the container component.

46. The metallic container component testing apparatus of claim 45 wherein an output signal from the controller to the first servo motor controls indexing of the container components.
